(19) United States
(12) Patent Application Publication
(10) Pub. No.: US 2022/0186188 A1
(43) Pub. Date: Jun. 16, 2022

(54) Method for Obtaining Pancreatic Progenitor Cells and Pancreatic Islet Beta Cells By Means of Differentiation of Human Pluripotent Stem Cells

(71) Applicant: Asia Regenerative Medicine, Ltd., Longhua District, Shenzhen (CN)

(72) Inventors: Wei Jiang, Wuhan (CN); Mengtian Tan, Wuhan (CN)

(73) Assignee: Asia Regenerative Medicine, Ltd., Longhua District, Shenzhen (CN)

(21) Appl. No.: 17/438,241

(22) PCT Filed: Mar. 12, 2020
(86) PCT No.: PCT/CN2020/078997

§ 371 Date: Sep. 10, 2021

(30) Foreign Application Priority Data
Mar. 13, 2019 (CN) 201910190210.4

(51) Int. Cl. C12N 5/071

(52) U.S. Cl. CPC C12N 5/0676; C12N 5/0678; C12N 2506/02; C12N 2501/415; C12N 2501/16; C12N 2501/117; C12N 2500/02

(57) ABSTRACT

Provided is a method for obtaining pancreatic precursor cells and islet β cells by differentiation of human pluripotent stem cells by adding a WNT signaling pathway inhibitor into the process of specialization of endoderm cells derived from human pluripotent stem cells into pancreatic lineage cells.

### TECHNICAL FIELD

The present disclosure belongs to the field of stem cells and regenerative medicine, and relates to a method for obtaining pancreatic progenitor cells and pancreatic islet β cells by differentiating human pluripotent stem cells.

### BACKGROUND

Diabetes is a metabolic disease characterized by elevated blood sugar. Whether insufficient insulin secretion caused by the lack of β cells due to immune damage (T1D) or elevated blood sugar caused by insulin resistance (T2D) will eventually cause β cell damage (Lam and Cherney, 2018). According to a report by the International Diabetes Federation (IDF) in 2017, there have been currently 425 million diabetic patients, and this large population of patients may increase to 629 million by 2045. Diabetes is no longer a health risk issue, and it has already become a global social crisis. Diabetes not only causes elevated blood sugar, but also leads to complications, which brings various burdens to the families and the society. At present, therapies for diabetes includes oral hypoglycemic drugs, insulin injections, insulin pumps, etc. Traditional therapies can only alleviate high blood sugar symptoms of diabetes, but cannot fundamentally cure diabetes. In 2000, Shapiro et al. carried out pancreatic islet isolation and transplantation, combined with appropriate immunosuppressive protocols, and achieved an insulin-independent efficacy (Shapiro et al., 2000), and excellent effects were also achieved in follow-up observations (McCall and Shapiro, 2012). Methods of pancreatic islet transplantation can fundamentally cure diabetes; however, due to the extreme lack of donors, the method has not become the main therapies for diabetes. Embryonic stem cells, as a type of stem cells with multi-directional differentiation potential, can be induced in vitro by associated small chemical molecules and cell growth factors to obtain relatively mature and functional tissues or organs. The method for obtaining tissues and organs by means of the directional differentiation of stem cells can also be used for the treatment of diabetes, thereby providing a new solution to the shortage of donors. It can be seen that how to obtain a large number of functional pancreatic islet β cells from embryonic stem cells will greatly benefit cell therapies of diabetes.

The expression levels of specific markers can be used for indicating the differentiation stage and maturity of cells. The characteristic transcription factor markers of pancreatic progenitor cells are co-expressed pancreatic duodenal homeobox 1 (PDX1) and NK6 homeobox protein 1 (NKX6-1), wherein the expression of PDX1 indicates the differentiation of the cells towards the pancreas, and the expression of PDX1 and NKX6-1 indicates the differentiation of the cells into pancreatic progenitor cells. They are key markers during the development of the human pancreas. The characteristic markers of pancreatic islet β cells are continuous expression of the transcription factor PDX1 and insulin (INS), and the co-expression of the transcription factor PDX1 and INS indicates the maturation of pancreatic islet β cells.

WNT signaling pathway is one of the most important signaling mechanisms in cells. Activation of this signaling pathway can cause the accumulation of β-catenin in the nucleus, and the binding of β-catenin to transcription factors such as TCF can regulate transcription and expression of the downstream gene, thereby affecting cell proliferation, differentiation, apoptosis, and migration. Therefore, the WNT signaling pathway also plays a vital role in early embryonic development and organ formation.

Due to the complexity of the differentiation into the pancreas and pancreatic islet β cells and the differences between various systems, it is difficult to obtain a reproducible and efficient protocol of pancreatic islet cell differentiation, and it is impossible to obtain a large number of stable pancreatic progenitor cells.

### SUMMARY

In order to solve the above-mentioned problems, the present disclosure provides an efficient method of obtaining pancreatic progenitor cells and pancreatic islet β cells by differentiating human pluripotent stem cells.

The present disclosure establishes an effective protocol of differentiation into pancreatic progenitor cells and further differentiation into pancreatic islet β cells by inhibiting the WNT signaling pathway during the differentiation stage of definitive endoderm into pancreatic progenitor cells.

A schematic diagram of the staged induction of the differentiating human pluripotent stem cells to obtain pancreatic progenitor cells and pancreatic islet β cells according to the present disclosure is shown in FIG. 1.

The specific technical solutions of the present disclosure are as follows:

1) differentiation of human pluripotent stem cells into definitive endoderm cells, involving:

a. preparing definitive endoderm stage medium 1, and culturing human pluripotent stem cells in the medium in a carbon dioxide incubator at 37 degrees Celsius for 1 day; and

b. preparing definitive endoderm stage medium 2, replacing the definitive endoderm stage medium 1 in above-mentioned step a with the definitive endoderm stage medium 2, and culturing the cells in a carbon dioxide incubator at 37 degrees Celsius for 3 days, during which the medium is replaced every day,

wherein the composition of the definitive endoderm stage medium 1 is: an IMDM medium and an F12 medium are mixed at a ratio of 1:1 as a basic medium, and the following components at the working concentrations are further included: 0.2% of bovine serum albumin (BSA), 1% of penicillin, 1% of streptomycin, 100 ng/ml of recombinant human activin-A (Activin A), and 50 ng/ml of Wnt3a protein, with the concentrations all being final concentrations; and

the composition of the definitive endoderm stage medium 2 is: an IMDM medium and an F12 medium are mixed at a ratio of 1:1 as a basic medium, and the following components at the working concentrations are further included: 0.2% of bovine serum albumin (BSA), 1% of penicillin, 1% of streptomycin, and 100 ng/ml of recombinant human activin-A (Activin A), with the concentrations all being final concentrations;

2) induction of the differentiation of definitive endoderm cells into pancreatic progenitor cells, involving:

preparing a pancreatic progenitor cell medium, replacing the medium in step 1) with the pancreatic progenitor cell medium, and culturing the cells in a carbon dioxide incubator at 37 degrees Celsius for 5 days, during which the medium is replaced every day,

wherein the composition of the pancreatic progenitor cell medium is: with an MCDB131 medium as a basic medium, and the following components at the working concentrations are further included: 0.5% of bovine serum albumin (BSA), 1.5 g/L of sodium bicarbonate, 1×glutamine (GlutaMAX), 10 mM glucose, 0.25 M vitamin C, 1×insulin-transferrin-selenium-ethanolamine additive (ITS-X), 50 ng/ml of fibroblast growth factor 7 (KGF), 0.5 μM SANT1 (with smo as an inhibitor target), 100 nM retinoic acid (TTNPB), 500 nM phorbol 12,13-dibutyrate (PDBu), 2 μM ALK inhibitor (K02288), and a WNT signaling pathway inhibitor (not added in a control group), with the concentrations all being final concentrations;

3) further differentiation of pancreatic progenitor cells to obtain pancreatic islet β cells, involving:

a. preparing pancreatic islet β cell medium 1, replacing the medium in step 2) with the pancreatic islet β cell medium 1, and culturing the cells in a carbon dioxide incubator at 37 degrees Celsius for 5 days, during which the medium is replaced every day; and

b. preparing pancreatic islet β cell medium 2, replacing the pancreatic islet β cell medium 1 in above-mentioned step a with the pancreatic islet β cell medium 2, and culturing the cells in a carbon dioxide incubator at 37 degrees Celsius for 5 days, during which the medium is replaced every day,

wherein the composition of the pancreatic islet β cell medium 1 is: with an MCDB131 medium as a basic medium, and the following components at the working concentrations are further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 2 μM ALK inhibitor (K02288), 1 μM triiodothyronine (T3), 10 μM YO-01027 (Notch signaling pathway inhibitor), and 10 μM zinc sulfate, with the concentrations all being final concentrations; and

the composition of the pancreatic islet β cell medium 2 is: with an MCDB131 medium as a basic medium, and the following components at the working concentrations are further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 1 μM triiodothyronine (T3), 10 μM Repsox (ALK5 inhibitor), 10 μM vitamin E, 10 μg/ml of heparin sodium, 2 μM R428 (Axl inhibitor), 10 μM zinc sulfate, and 10 mM N-acetyl-L-cysteine (N-cys), with the concentrations all being final concentrations.

The human pluripotent stem cells include human embryonic stem cells and human induced pluripotent stem cells.

In some embodiments, the WNT signaling pathway inhibitor in step 2) is XAV-939 or IWR-1.

In some embodiments, the concentration of the WNT signaling pathway inhibitor in step 2) is 2 μM.

During the differentiation of human pluripotent stem cells into pancreatic progenitor cells in the disclosure, the cell growth is not affected after the WNT signaling pathway inhibitor (abbreviated as WNT inhibitor) is added. After immunofluorescence staining, flow cytometry, and fluorescence quantitative PCR analysis, a considerable amount of PDX1-positive pancreatic progenitor cells are obtained, and the expression of the two transcription factors PDX1 and NKX6-1 of the pancreatic progenitor cells is significantly increased; in addition, after the step of further differentiation into pancreatic islet β cells, pancreatic islet β cells expressing the insulin (abbreviated as INS) are obtained. Fluorescence quantitative PCR analysis has shown that the transcription level of the insulin gene INS in the pancreatic islet β cells is greatly increased, and immunofluorescence staining has revealed co-staining of PDX1 protein (nuclear localization) and INS protein (cytoplasmic localization) in the pancreatic islet β cells. DAPI is a nuclear dye used for marking the total number of cells.

During the differentiation of definitive endoderm cells into pancreatic progenitor cells, a variety of small molecules/growth factors including a WNT signaling pathway inhibitor are added, wherein the WNT signaling pathway inhibitor inhibits the WNT signaling pathway, induce the effective differentiation of the human pluripotent stem cells into pancreatic progenitor cells and further into pancreatic islet β cells, thereby providing a favorable solution to obtain pancreatic islet β cells in vitro for the cell therapy of diabetes.

### BRIEF DESCRIPTION OF DRAWINGS

FIG. 1 is a schematic diagram of the staged induction of the differentiation of human pluripotent stem cells to obtain pancreatic progenitor cells and pancreatic islet β cells according to the present disclosure.

FIG. 2 shows the detection results of the number of PDX1-positive pancreatic progenitor cells by means of flow cytometry in Embodiment 1 of the present disclosure, wherein the negative control group is a negative control in which no primary antibody is added; the WNT-inhibitor-free group is a control group in which a primary antibody is added and no WNT signaling pathway inhibitor XAV-939 is added; and the WNT inhibitor group is a positive group in which the primary antibody and the WNT signaling pathway inhibitor XAV-939 are added.

FIG. 3 shows the relative expression results of mRNAs of PDX1 and NKX6-1 in pancreatic progenitor cells by real-time fluorescence quantitative PCR analysis in Embodiment 1 of the present disclosure.

FIG. 4 shows the results of immunofluorescence staining analysis of PDX1 in pancreatic progenitor cells in Embodiment 1 of the present disclosure, wherein PDX1-positive cells are marked by green fluorescence; and the total number of cells is marked by blue DAPI.

FIG. 5 shows the relative expression results of mRNA of INS in pancreatic islet β cells by real-time fluorescence quantitative PCR analysis in Embodiment 1 of the present disclosure.

FIG. 6 shows the results of immunofluorescence staining analysis of PDX1/INS in pancreatic islet β cells in Embodiment 1 of the present disclosure, wherein PDX1-positive cells are marked by green fluorescence; INS-positive cells are marked by red fluorescence; and the total number of cells is marked by blue DAPI.

FIG. 7 shows the detection results of the number of PDX1-positive pancreatic progenitor cells by means of flow cytometry in Embodiment 2 of the present disclosure, wherein the negative control group is a negative control in which no primary antibody is added; the WNT-inhibitor-free group that is a control group in which a primary antibody is added and no WNT signaling pathway inhibitor IWR-1 is added; and the WNT inhibitor group is a positive group in which the primary antibody and the WNT signaling pathway inhibitor IWR-1 are added.

FIG. 8 shows the relative expression results of mRNAs of PDX1 and NKX6-1 in pancreatic progenitor cells by real-time fluorescence quantitative PCR analysis in Embodiment 2 of the present disclosure.

FIG. 9 shows the results of immunofluorescence staining analysis of PDX1 in pancreatic progenitor cells in Embodiment 2 of the present disclosure, wherein PDX1-positive cells are marked by green fluorescence; the total number of cells is marked by blue DAPI.

FIG. 10 shows the relative expression results of mRNA of INS in pancreatic islet β cells by real-time fluorescence quantitative PCR analysis in Embodiment 2 of the present disclosure.

FIG. 11 shows the results of immunofluorescence staining analysis of PDX1/INS in pancreatic islet β cells in Embodiment 2 of the present disclosure, wherein PDX1-positive cells are marked by green fluorescence; INS-positive cells are marked by red fluorescence; and the total number of cells is marked by blue DAPI.

FIG. 12 shows the detection results of the number of PDX1-positive pancreatic progenitor cells by means of flow cytometry in Embodiment 3 of the present disclosure, wherein the negative control group is a negative control in which no primary antibody is added; the WNT-inhibitor-free group is a control group in which a primary antibody is added and no WNT signaling pathway inhibitor XAV-939 is added; and the WNT inhibitor group is a positive group in which the primary antibody and the WNT signaling pathway inhibitor XAV-939 are added.

FIG. 13 shows the relative expression results of mRNAs of PDX1 and NKX6-1 in pancreatic progenitor cells by real-time fluorescence quantitative PCR analysis in Embodiment 3 of the present disclosure.

FIG. 14 shows the results of immunofluorescence staining analysis of PDX1 in pancreatic progenitor cells in Embodiment 3 of the present disclosure, wherein PDX1-positive cells are marked by green fluorescence; the total number of cells is marked by blue DAPI.

FIG. 15 shows the relative expression results of mRNA of INS in pancreatic islet β cells by real-time fluorescence quantitative PCR analysis in Embodiment 3 of the present disclosure.

FIG. 16 shows the results of immunofluorescence staining analysis of PDX1/INS in pancreatic islet β cells in Embodiment 3 of the present disclosure, wherein PDX1-positive cells are marked by green fluorescence; INS-positive cells are marked by red fluorescence; and the total number of cells is marked by blue DAPI.

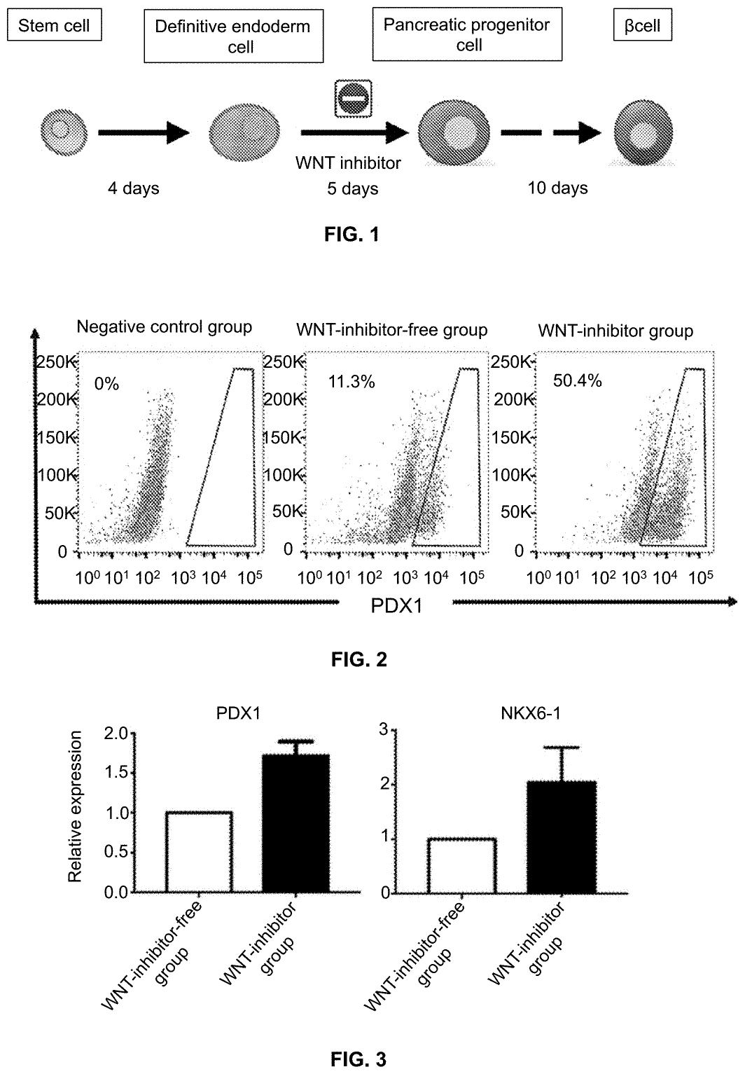

### DETAILED DESCRIPTION

The present disclosure is further described below with the accompanying drawings and specific embodiments, and the embodiments are only illustrative of the method, and do not limit the rest content disclosed by the present invention in any way.

Unless otherwise specified, the test methods in the following embodiments are all conventional methods.

Unless otherwise specified, the materials, reagents, etc. in the following embodiments can all be obtained from commercial sources.

### Example 1 Differentiation of Human Embryonic Stem Cells into Pancreatic Progenitor Cells and Mature Pancreatic Islet β Cells by Induction of the WNT Signaling Pathway Inhibitor XAV-939

(1) Cell Differentiation

1) Differentiation of Human Embryonic Stem Cells into Definitive Endoderm Cells:

a. definitive endoderm stage medium 1 was prepared, and the human embryonic stem cells were cultured in the medium in a carbon dioxide incubator at 37 degrees Celsius for 1 day; and

b. definitive endoderm stage medium 2 was prepared, the definitive endoderm stage medium 1 in above-mentioned step a was replaced with the definitive endoderm stage medium 2, and the cells were cultured in a carbon dioxide incubator at 37 degrees Celsius for 3 days, during which the medium was replaced every day, wherein the composition of the definitive endoderm stage medium 1 was: an IMDM medium and an F12 medium were mixed at a ratio of 1:1 as a basic medium, and the following components at the working concentrations were further included: 0.2% of bovine serum albumin (BSA), 1% of penicillin, 1% of streptomycin, 100 ng/ml of recombinant human activin-A (Activin A), and 50 ng/ml of Wnt3a protein, with the concentrations all being final concentrations; and the composition of the definitive endoderm stage medium 2 was: an IMDM medium and an F12 medium were mixed at a ratio of 1:1 as a basic medium, and the following components at the working concentrations were further included: 0.2% of bovine serum albumin (BSA), 1% of penicillin, 1% of streptomycin, and 100 ng/ml of recombinant human activin-A (Activin A), with the concentrations all being final concentrations.

2) Induction of the Differentiation of Definitive Endoderm Cells into Pancreatic Progenitor Cells:

a pancreatic progenitor cell medium was prepared, the medium in step 1) was replaced with the pancreatic progenitor cell medium, and the cells were cultured in a carbon dioxide incubator at 37 degrees Celsius for 5 days, during which the medium was replaced every day, wherein the composition of the pancreatic progenitor cell medium was: with an MCDB131 medium as a basic medium, and the following components at the working concentrations were further included: 0.5% of bovine serum albumin (BSA), 1.5 g/L of sodium bicarbonate, 1×glutamine (GlutaMAX), 10 mM glucose, 0.25 M vitamin C, 1×insulin-transferrin-selenium-ethanolamine additive (ITS-X), 50 ng/ml of fibroblast growth factor 7 (KGF), 0.5 μM SANT1 (with smo as an inhibitor target), 100 nM retinoic acid (TTNPB), 500 nM phorbol 12,13-dibutyrate (PDBu), 2 μM ALK inhibitor (K02288), and 2 μM WNT signaling pathway inhibitor XAV-939 (not added in a control group), with the concentrations all being final concentrations.

3) Further Differentiation of Pancreatic Progenitor Cells to Obtain Pancreatic Islet β Cells:

a. pancreatic islet β cell medium 1 was prepared, the medium in step 2) was replaced with the pancreatic islet β cell medium 1, and the cells were cultured in a carbon dioxide incubator at 37 degrees Celsius for 5 days, during which the medium was replaced every day; and

b. pancreatic islet β cell medium 2 was prepared, the pancreatic islet β cell medium 1 in above-mentioned step a was replaced with the pancreatic islet β cell medium 2, and the cells were cultured in a carbon dioxide incubator at 37 degrees Celsius for 5 days, during which the medium was replaced every day, wherein the composition of the pancreatic islet β cell medium 1 was: with an MCDB131 medium as a basic medium, and the following components at the working concentrations were further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 2 μM ALK inhibitor (K02288), 1 μM triiodothyronine (T3), 10 μM YO-01027 (Notch signaling pathway inhibitor), and 10 μM zinc sulfate, with the concentrations all being final concentrations; and the composition of the pancreatic islet β cell medium 2 was: with an MCDB131 medium as a basic medium, and the following components at the working concentrations were further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 1 μM triiodothyronine (T3), 10 μM Repsox (ALK5 inhibitor), 10 μM vitamin E, 10 μg/ml of heparin sodium, 2 μM R428 (Axl inhibitor), 10 μM zinc sulfate, and 10 mM N-acetyl-L-cysteine (N-cys), with the concentrations all being final concentrations.

(2) Detection of the Expression of PDX1 in the Pancreatic Progenitor Cell Stage by Means of Flow Cytometry

Adherent cells cultured on a pancreatic progenitor cell stage culture plate were washed three times with PBS free of calcium and magnesium ions (i.e., DPBS), the remaining medium was removed, the cells were covered with 0.05% of chymotrypsin-trypsin (Trypsin) and placed in an incubator at 37° C. for 10 minutes of digestion, the digestion was then terminated using DPBS containing 2% fetal bovine serum (FBS), the product was carefully pipetted to form a single cell suspension, the single cell suspension was centrifuged at 3000 rpm for 5 minutes, and the supernatant was removed to leave cell pellets; the cell pellets were resuspended with 2% FBS in DPBS, the product was carefully pipetted until uniformly mixed, and centrifuged at 3000 rpm for 5 minutes, the supernatant was removed to leave cell pellets, and the operation was repeated once again. A perforating agent in a cell transcription factor kit was prepared (prepared immediately before use, and attention should be paid to avoid light), 1 ml of the perforating agent was used for gentle pipetting, the cell pellets were resuspended, and the product was placed on ice in the dark for 40 minutes of perforation; the perforation was terminated using 1 ml of a detergent in the cell transcription factor perforation kit which was prepared in advance, then the product was centrifuged at 3000 rpm for 5 minutes, and the supernatant was removed to leave cell pellets; 1 ml of the detergent was added to the pellets to resuspend the cells, the product was centrifuged at 3000 rpm for 5 minutes, the supernatant was removed to leave cell pellets, and the operation was repeated once again. A primary antibody diluted to a working concentration with the detergent was added, and the product was placed on a rotary mixer at 4° C. overnight; the next day, the product was taken out from 4° C. and rewarmed for 30 minutes, the primary antibody was terminated using 1 ml of the detergent, then the product was centrifuged at 3000 rpm for 5 minutes, and the supernatant was removed to leave cell pellets; 1 ml of the detergent was added to the cell pellets, the product was centrifuged at 3000 rpm for 5 minutes, the supernatant was removed to leave cell pellets, and the operation was repeated once again to leave the pellets, wherein close observation was carried out each time the supernatant was aspirated, and the cell pellets should not be aspirated. A fluorescent secondary antibody diluted with the detergent was added, and the product was placed on a rotary mixer at room temperature and incubated for 2 hours; the secondary antibody was terminated using 1 ml of the detergent, then the product was centrifuged at 3000 rpm for 5 minutes, and the supernatant was removed to leave cell pellets; 1 ml of the detergent was added to the cell pellets, the product was centrifuged at 3000 rpm for 5 minutes, the supernatant was removed to leave the cell pellets, and the operation was repeated once again to leave the pellets. The cell pellets were gently pipetted using 200 μl of DPBS and transferred to a flow tube; and an FACSCelesta flow cytometric analyzer was used for sample analysis. The primary antibody used was human PDX-1/IPF1 antibody (AF2419, 1:500, RD); and the secondary antibody used in this study was donkey anti-goat PE fluorescent dye (115-035-003, 1:200, Jackson ImmunoResearch).

The expression of PDX1 in the pancreatic progenitor cell stage was detected by means of flow cytometry, and the results were as shown in FIG. 2. Compared with a control group in which no WNT signaling pathway inhibitor was added, PDX1-positive pancreatic progenitor cells were significantly increased after the WNT signaling pathway inhibitor XAV-939 was added.

(3) Detection of the relative expression of mRNAs of PDX1 and NKX6-1 in the pancreatic progenitor cell stage and detection of the relative expression of mRNA of INS in the mature pancreatic islet β cell stage by Real-time fluorescence quantitative PCR

1) Total RNA Extraction from Cells

Cells cultured in a 24-well plate were collected, and washed twice with 500 μl of DPBS at first, then covered with 200 μl of chymotrypsin-trypsin (Trypsin), and placed at 37° C. for 5 minutes of digestion. Whether the cells float in pieces was observed, and the digestion was terminated using 500 μl of DMEM/F12 medium, then the product was centrifuged at 1000 rpm for 3 minutes, the supernatant was removed, and cell pellets were collected and added to a new RNase-free centrifuge tube. A lysis solution in a rapid and small-amount extraction kit of total RNA was used, and 350 μl of the lysis solution was added to each tube and then uniformly mixed using a pipette (to avoid bubble generation caused by excessive pipetting), or a vortex mixer was used to break up the cells such that the cells were fully lysed; the product was centrifuged at 14,000×g for 5 minutes at room temperature; after centrifugation, the liquid was taken and added to an equal volume of an RNA binding agent in which anhydrous ethanol was added in advance, then the mixture was added to each centrifuge tube, pipetted five times with a pipette, and the liquid, which had been pipetted until uniformly mixed, was transferred to a high-purification RNA column, which was mounted in a 2 ml collection tube for 1 minute of centrifugation at 12,000×g; the filtrate was discarded, the column was placed back into the collection tube, 500 μl of kit detergent 1 was added to the column, centrifugation was performed at 12,000×g for 1 minute, the filtrate was discarded, the column was placed back into the collection tube, then 500 μl of kit detergent 2 (diluted with ethanol) was added, centrifugation was performed at 12,000×g for 1 minute, and 500 μl of kit detergent 2 was added so as to repeat the operation once again; the filtrate was discarded, and the column was placed back into the collection tube for 2 minutes of centrifugation at 12,000×g; then the column was transferred to a new 1.5 ml centrifuge tube, 50 μl of RNase-free water was added to the center of a column membrane, which was left to stand at room temperature for 2 minutes, followed by 1 minute of centrifugation at 12,000×g, and the above-mentioned operation was repeated once again to improve the RNA elution efficiency. The column was discarded and the RNA was stored in an ultra-low-temperature refrigerator at −80° C.

2) Preparation of cDNA

The concentration and purity of the total RNA extracted above was determined (A260/A280 absorbance ratio >1.8), and a reverse transcription kit from the manufacturer Biotool was used to obtain cDNA: wherein the total RNA was taken and placed on ice, the volume required for the reverse transcription of 1 μg of RNA was calculated according to the concentration, and the corresponding volume of RNA was added; 4 μl of 5×qRT reverse transcription mix was then added, and the rest was made up with RNase-free water to obtain a total volume of 20 μl of a reverse transcription mixture liquid; and the reverse transcription mixture liquid was gently mixed, the uniformly mixed liquid was centrifuged by means of a palm centrifuge to the bottom of a 1.5 ml centrifuge tube, a common Bio-rad PCR instrument was used for reverse transcription under the reaction conditions of 25° C. for 10 min, 42° C. for 30 min and 85° C. for 5 min, and afterwards, the product was stored at −20° C.

3) Detection of the Relative Expression of mRNAs of PDX1, NKX6-1 and INS by SYBR Green Real-Time Fluorescence Quantitative PCR

50 ng of cDNA obtained from the above-mentioned reverse transcription was taken, 5 μl of 2×SYBR Green qPCR mixture, 5 μM of upstream primer and 5 μM of downstream primer were added, and the rest was made up with RNase-free water to a total volume of 10 μl; and CFX384 Bio-rad model quantitative PCR instrument was used for amplification under the reaction conditions of 95° C. for 5 min, 95° C. for 15 s and 60° C. for 30 s, repeated 39 cycles, 95° C. for 15 s, and ending with 65° C. for 15 s. The experimental results were analyzed using a ΔΔCT method with reference to the housekeeping gene GAPDH. Primers used in this study were: GAPDH upstream primer AATGAAGGGGTCATTGATGG, downstream primer AAGGTGAAGGTCGGA GTCAA; PDX1 upstream primer TTAGGATGTGGACGTAATTCCTGTT, downstream primer GGCCACTGT GCTTGTCTTCA; NKX6-1 upstream primer AGACCCACTTTT TCCGGACA, downstream primer CCAACGAATAGGCCAAACGA; INSMLIN upstream primer GCAGCCTTTGTGAACCAACAC, and downstream primer CCCCG CACACTAGGTAGAGA.

The relative expression of mRNAs of PDX1 and NKX6-1 in the pancreatic progenitor cell stage was detected by means of real-time fluorescent quantitative PCR, and the results were as shown in FIG. 3, wherein in the stage of differentiation from definitive endoderm cells to pancreatic progenitor cells, the mRNAs of the marker PDX1 and its downstream gene NKX6-1 were significantly increased in the pancreatic progenitor cells after the adding of the WNT signaling pathway inhibitor XAV-939; and

the relative expression of mRNA of INS in the mature pancreatic islet β cell stage was detected by means of real-time fluorescent quantitative PCR, and the results were as shown in FIG. 5, wherein in the process of further differentiation from pancreatic progenitor cells into mature pancreatic islet β cells, the transcription level of the insulin gene INS in the mature pancreatic islet β cell stage was greatly increased.

(4) Immunofluorescence Detection of the Expression of PDX1 Protein in the Pancreatic Progenitor Cell Stage and PDX1/INS Proteins in the Mature Pancreatic Islet β Cell Stage

Cells in a culture plate were washed three times with a phosphate buffered saline (PBS), 3 minutes each time; the cells were fixed with 4% paraformaldehyde at room temperature for 20 minutes, and washed three times with PBS, 3 minutes each time; a blocking solution was prepared in advance (by adding Triton at a final concentration of 0.3% to 9 ml of DPBS, adding donkey serum at a final concentration of 10%, and uniformly mixing); the above-prepared blocking solution was used for blocking and perforation at room temperature for 2 hours; the blocking solution was soaked up, a working concentration of primary antibody prepared with the blocking solution was then added, and the product was left to stand at room temperature for 30 minutes and then placed in a refrigerator at 4° C. overnight; the next day, the product was washed three times with PBS, 3 minutes each time; a fluorescent secondary antibody of donkey serum was added and incubation was carried out at room temperature for 2 hours; in the same way, the product was washed three times with PBS, 3 minutes each time; the product was incubated with a working solution of the nuclear dye DAPI at room temperature for 10 minutes, and washed three times with PBS, 3 minutes each time; then a fluorescence microscope was used for observation and data were collected. The primary antibodies used in this study were as follows: human PDX-1/IPF1 antibody (AF2419, 1:1000, RD); Anti-PDX1 (ab47267, 1:200, Abcam); Anti-PDX1 (ab47383, 1:1000, Abcam); and insulin antibody (SC-29062, 1:100, SantaCruz). The fluorescent secondary antibodies used in this study were as follows: donkey anti-goat TRITC fluorescent dye (705-025-147, 1:200, Jackson ImmunoResearch); donkey anti-goat FITC fluorescent dye (705-095-003, 1:200, Jackson ImmunoResearch); donkey anti-goat 488 fluorescent dye (705-545-147, 1:200, Jackson ImmunoResearch); donkey anti-rabbit TRITC fluorescent dye (711-025-152, 1:200, Jackson ImmunoResearch); donkey anti-rabbit 488 fluorescent dye (705-095-152, 1:200, Jackson ImmunoResearch); donkey anti-mouse FITC fluorescent dye (715-095-150, 1:200, Jackson ImmunoResearch); and nuclear dye DAPI (Roche, USA).

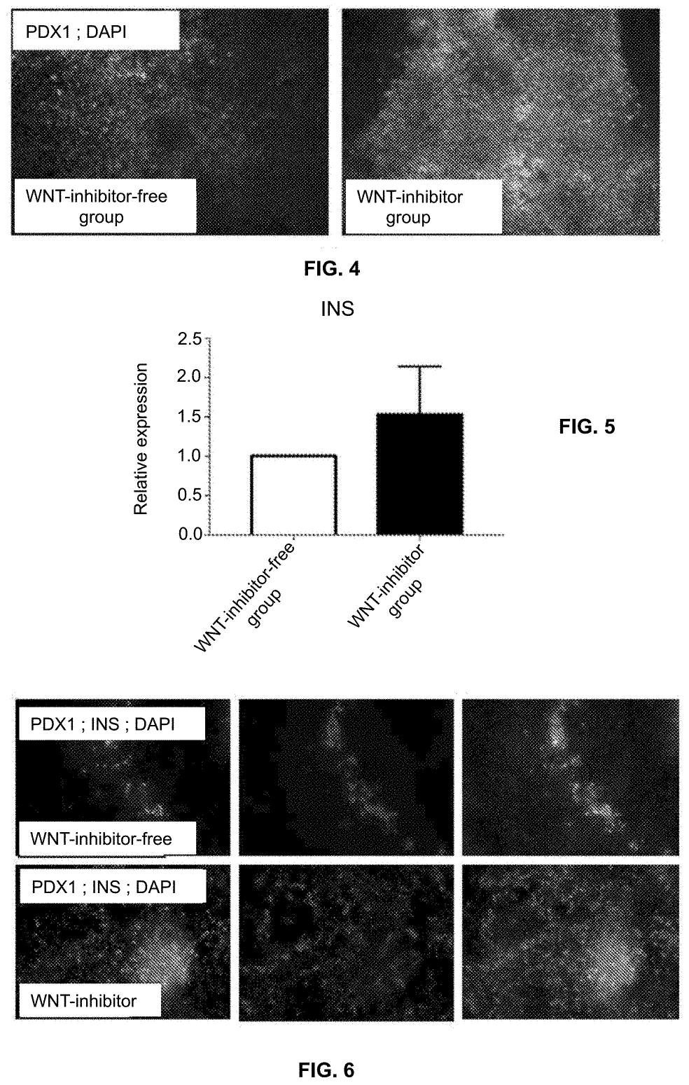

The expression of PDX1 protein in the pancreatic progenitor cell stage was detected by means of immunofluorescence, and the results were as shown in FIG. 4, wherein PDX1-positive pancreatic progenitor cells were marked by green fluorescence, and blue DAPI indicated the total cell number of pancreatic progenitor cells. It could be seen that the adding of the WNT signaling pathway inhibitor did not affect the cell growth, and the PDX1-positive pancreatic progenitor cells were significantly increased after the WNT signaling pathway inhibitor XAV-939 was added.

The expression of PDX1/INS proteins in the mature pancreatic islet β cell stage was detected by means of immunofluorescence, and the results were as shown in FIG. 6, wherein PDX1-positive pancreatic islet β cells were marked by green fluorescence, INS-positive pancreatic islet β cells were marked by red fluorescence, and blue DAPI indicated the total cell number of pancreatic islet β cells. It could be seen that after the adding of the WNT signaling pathway inhibitor XAV-939 in the pancreatic progenitor cell stage, the number of insulin INS-positive and PDX1 co-stained pancreatic islet β cells in the mature pancreatic islet β cell stage was significantly increased.

The results showed that the adding of the staged WNT signaling pathway inhibitor provided by the present disclosure could greatly increase the number of mature pancreatic islet β cells.

### Example 2 Differentiation of Human Embryonic Stem Cells into Pancreatic Progenitor Cells and Pancreatic Islet β Cells by Induction of the WNT Signaling Pathway Inhibitor IWR-1

(1) Cell Differentiation

1) Differentiation of Human Embryonic Stem Cells into Definitive Endoderm Cells:

a. definitive endoderm stage medium 1 was prepared, and the human embryonic stem cells were cultured in the medium in a carbon dioxide incubator at 37 degrees Celsius for 1 day; and

b. definitive endoderm stage medium 2 was prepared, the definitive endoderm stage medium 1 in above-mentioned step a was replaced with the definitive endoderm stage medium 2, and the cells were cultured in a carbon dioxide incubator at 37 degrees Celsius for 3 days, during which the medium was replaced every day, wherein the composition of the definitive endoderm stage medium 1 was: a basic medium was prepared by mixing an IMDM medium and an F12 medium at a ratio of 1:1, and the following components at the working concentrations were further included: 0.2% of bovine serum albumin (BSA), 1% of penicillin, 1% of streptomycin, 100 ng/ml of recombinant human activin-A (Activin A), and 50 ng/ml of Wnt3a protein, with the concentrations all being final concentrations; and the composition of the definitive endoderm stage medium 2 was: a basic medium was prepared by mixing an IMDM medium and an F12 medium at a ratio of 1:1, and the following components at the working concentrations were further included: 0.2% of bovine serum albumin (BSA), 1% of penicillin, 1% of streptomycin, and 100 ng/ml of recombinant human activin-A (Activin A), with the concentrations all being final concentrations.

2) Induction of the Differentiation of Definitive Endoderm Cells into Pancreatic Progenitor Cells:

a pancreatic progenitor cell medium was prepared, the medium in step 1) was replaced with the pancreatic progenitor cell medium, and the cells were cultured in a carbon dioxide incubator at 37 degrees Celsius for 5 days, during which the medium was replaced every day, wherein the composition of the pancreatic progenitor cell medium was: with an MCDB131 medium as a basic medium, and the following components at the working concentrations were further included: 0.5% of bovine serum albumin (BSA), 1.5 g/L of sodium bicarbonate, 1×glutamine (GlutaMAX), 10 mM glucose, 0.25 M vitamin C, 1×insulin-transferrin-selenium-ethanolamine additive (ITS-X), 50 ng/ml of fibroblast growth factor 7 (KGF), 0.5 μM SANT1 (with smo as an inhibitor target), 100 nM retinoic acid (TTNPB), 500 nM phorbol 12,13-dibutyrate (PDBu), 2 μM ALK inhibitor (K02288), and 2 μM WNT signaling pathway inhibitor IWR-1 (not added in a control group), with the concentrations all being final concentrations.

3) Further Differentiation of Pancreatic Progenitor Cells to Obtain Pancreatic Islet β Cells:

a. pancreatic islet β cell medium 1 was prepared, the medium in step 2) was replaced with the pancreatic islet β cell medium 1, and the cells were cultured in a carbon dioxide incubator at 37 degrees Celsius for 5 days, during which the medium was replaced every day; and

b. pancreatic islet β cell medium 2 was prepared, the pancreatic islet β cell medium 1 in above-mentioned step a was replaced with the pancreatic islet β cell medium 2, and the cells were cultured in a carbon dioxide incubator at 37 degrees Celsius for 5 days, during which the medium was replaced every day, wherein the composition of the pancreatic islet β cell medium 1 was: with an MCDB131 medium as a basic medium, and the following components at the working concentrations were further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 2 μM ALK inhibitor (K02288), 1 μM triiodothyronine (T3), 10 μM YO-01027 (Notch signaling pathway inhibitor), and 10 μM zinc sulfate, with the concentrations all being final concentrations; and the composition of the pancreatic islet β cell medium 2 was: with an MCDB131 medium as a basic medium, and the following components at the working concentrations were further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 1 μM triiodothyronine (T3), 10 μM Repsox (ALK5 inhibitor), 10 μM vitamin E, 10 μg/ml of heparin sodium, 2 μM R428 (Axl inhibitor), 10 μM zinc sulfate, and 10 mM N-acetyl-L-cysteine (N-cys), with the concentrations all being final concentrations.

(2) Detection of the Expression of PDX1 in the Pancreatic Progenitor Cell Stage by Means of Flow Cytometry

Adherent cells cultured on a pancreatic progenitor cell stage culture plate were washed three times with PBS free of calcium and magnesium ions (i.e., DPBS), the remaining medium was removed, the cells were covered with 0.05% of chymotrypsin-trypsin (Trypsin) and placed in an incubator at 37° C. for 10 minutes of digestion, the digestion was then terminated using DPBS containing 2% fetal bovine serum (FBS), the product was carefully pipetted to form a single cell suspension, the single cell suspension was centrifuged at 3000 rpm for 5 minutes, and the supernatant was removed to leave cell pellets; the cell pellets were resuspended with 2% FBS in DPBS, the product was carefully pipetted until uniformly mixed, and centrifuged at 3000 rpm for 5 minutes, the supernatant was removed to leave cell pellets, and the operation was repeated once again. A perforating agent in a cell transcription factor kit was prepared (prepared immediately before use, and attention should be paid to avoid light), 1 ml of the perforating agent was used for gentle pipetting, the cell pellets were resuspended, and the product was placed on ice in the dark for 40 minutes of perforation; the perforation was terminated using 1 ml of a detergent in the cell transcription factor perforation kit which was prepared in advance, then the product was centrifuged at 3000 rpm for 5 minutes, and the supernatant was removed to leave cell pellets; 1 ml of the detergent was added to the pellets to resuspend the cells, the product was centrifuged at 3000 rpm for 5 minutes, the supernatant was removed to leave cell pellets, and the operation was repeated once again. A primary antibody diluted to a working concentration with the detergent was added, and the product was placed on a rotary mixer at 4° C. overnight; the next day, the product was taken out from 4° C. and rewarmed for 30 minutes, the primary antibody was terminated using 1 ml of the detergent, then the product was centrifuged at 3000 rpm for 5 minutes, and the supernatant was removed to leave cell pellets; 1 ml of the detergent was added to the cell pellets, the product was centrifuged at 3000 rpm for 5 minutes, the supernatant was removed to leave cell pellets, and the operation was repeated once again to leave the pellets, wherein close observation was carried out each time the supernatant was aspirated, and the cell pellets should not be aspirated. A fluorescent secondary antibody diluted with the detergent was added, and the product was placed on a rotary mixer at room temperature and incubated for 2 hours; the secondary antibody was terminated using 1 ml of the detergent, then the product was centrifuged at 3000 rpm for 5 minutes, and the supernatant was removed to leave cell pellets; 1 ml of the detergent was added to the cell pellets, the product was centrifuged at 3000 rpm for 5 minutes, the supernatant was removed to leave cell pellets, and the operation was repeated once again to leave the pellets; The cell pellets were gently pipetted using 200 μl of DPBS and transferred to a flow tube; and an FACSCelesta flow cytometric analyzer was used for sample analysis. The primary antibody used was human PDX-1/IPF1 antibody (AF2419, 1:500, RD); and the secondary antibody used in this study was donkey anti-goat PE fluorescent dye (115-035-003, 1:200, Jackson ImmunoResearch).

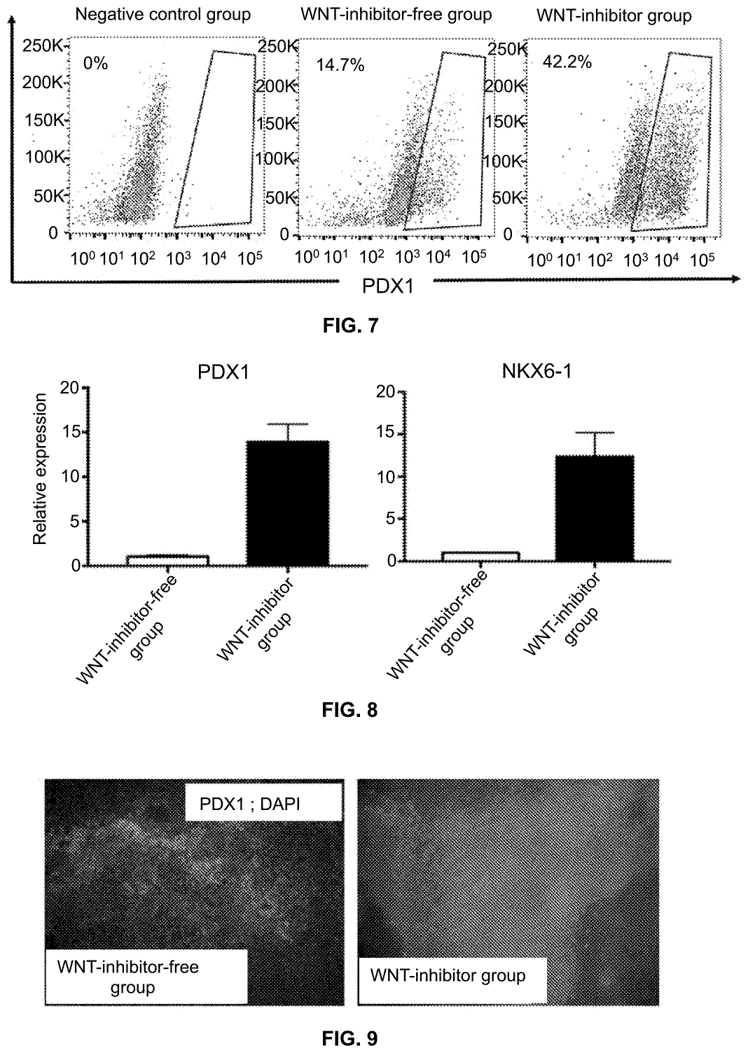

The expression of PDX1 in the pancreatic progenitor cell stage was detected by means of flow cytometry, and the results were as shown in FIG. 7. Compared with a control group in which no WNT signaling pathway inhibitor was added, PDX1-positive pancreatic progenitor cells were significantly increased after the WNT signaling pathway inhibitor IWR-1 was added.

(3) Detection of the Relative Expression of mRNAs of PDX1 and NKX6-1 in the Pancreatic Progenitor Cell Stage and Detection of the Relative Expression of mRNA of INS in the Mature Pancreatic Islet β Cell Stage by Real-Time Fluorescence Quantitative PCR

1) Total RNA Extraction from Cells

Cells cultured in a 24-well plate were collected, and washed twice with 500 μl of DPBS at first, then covered with 200 μl of chymotrypsin-trypsin (Trypsin), and placed at 37° C. for 5 minutes of digestion. Whether the cells float in pieces was observed, and the digestion was terminated using 500 μl of DMEM/F12 medium, then the product was centrifuged at 1000 rpm for 3 minutes, the supernatant was removed, and cell pellets were collected and added to a new RNase-free centrifuge tube. A lysis solution in a rapid and small-amount extraction kit of total RNA was used, and 350 μl of the lysis solution was added to each tube and then uniformly mixed using a pipette (to avoid bubble generation caused by excessive pipetting), or a vortex mixer was used to break up the cells such that the cells were fully lysed; the product was centrifuged at 14,000×g for 5 minutes at room temperature; after centrifugation, a liquid was taken and added to an equal volume of an RNA binding agent in which anhydrous ethanol was added in advance, the mixture was added to each centrifuge tube, pipetted five times with a pipette, and the liquid, which had been pipetted until uniformly mixed, was transferred to a high-purification RNA column, which was mounted in a 2 ml collection tube for 1 minute of centrifugation at 12,000×g; the filtrate was discarded, the column was placed back into the collection tube, 500 μl of kit detergent 1 was added to the column, centrifugation was performed at 12,000×g for 1 minute, the filtrate was discarded, the column was placed back into the collection tube, then 500 μl of kit detergent 2 (diluted with ethanol) was added, centrifugation was performed at 12,000×g for 1 minute, and 500 μl of kit detergent 2 was added so as to repeat the operation once again; the filtrate was discarded, and the column was placed back into the collection tube for 2 minutes of centrifugation at 12,000×g; then the column was transferred to a new 1.5 ml centrifuge tube, 50 μl of RNase-free water was added to the center of a column membrane, which was left to stand at room temperature for 2 minutes, followed by 1 minute of centrifugation at 12,000×g, and the above-mentioned operation was repeated once again to improve the RNA elution efficiency. The column was discarded and the RNA was stored in an ultra-low-temperature refrigerator at −80° C.

2) Preparation of cDNA

The concentration and purity of the total RNA extracted above was determined (A260/A280 absorbance ratio >1.8), and a reverse transcription kit from the manufacturer Biotool was used to obtain cDNA, wherein the total RNA was taken and placed on ice, the volume required for the reverse transcription of 1 μg of RNA was calculated according to the concentration, and the corresponding volume of RNA was added; 4 μl of 5×qRT reverse transcription mix was then added, and the rest was made up with RNase-free water to obtain a total volume of 20 μl of a reverse transcription mixture liquid; and the reverse transcription mixture liquid was gently mixed, the uniformly mixed liquid was centrifuged by means of a palm centrifuge to the bottom of a 1.5 ml centrifuge tube, a common Bio-rad PCR instrument was used for reverse transcription under the reaction conditions of 25° C. for 10 min, 42° C. for 30 min and 85° C. for 5 min, and afterwards, the product was stored at −20° C.

3) Detection of the Relative Expression of mRNAs of PDX1, NKX6-1 and INS by SYBR Green Real-Time Fluorescence Quantitative PCR

50 ng of cDNA obtained from the above-mentioned reverse transcription was taken, 5 μl of 2×SYBR Green qPCR mixture, 5 μM of upstream primer and 5 μM of downstream primer were added, and the rest was made up with RNase-free water to a total volume of 10 μl; and CFX384 Bio-rad model quantitative PCR instrument was used for amplification under the reaction conditions of 95° C. for 5 min, 95° C. for 15 s and 60° C. for 30 s, repeated 39 cycles, 95° C. for 15 s, and ending with 65° C. for 15 s. The experimental results were analyzed using a ΔΔCT method with reference to the housekeeping gene GAPDH. Primers used in this study were: GAPDH upstream primer AATGAAGGGGTCATTGATGG, downstream primer AAGGTGAAGGTCGGA GTCAA; PDX1 upstream primer TTAGGATGTGGACGTAATTCCTGTT, downstream primer GGCCACTGT GCTTGTCTTCA; NKX6-1 upstream primer AGACCCACTTTT TCCGGACA, downstream primer CCAACGAATAGGCCAAACGA; INSMLIN upstream primer GCAGCCTTTGTGAACCAACAC, and downstream primer CCCCG CACACTAGGTAGAGA.

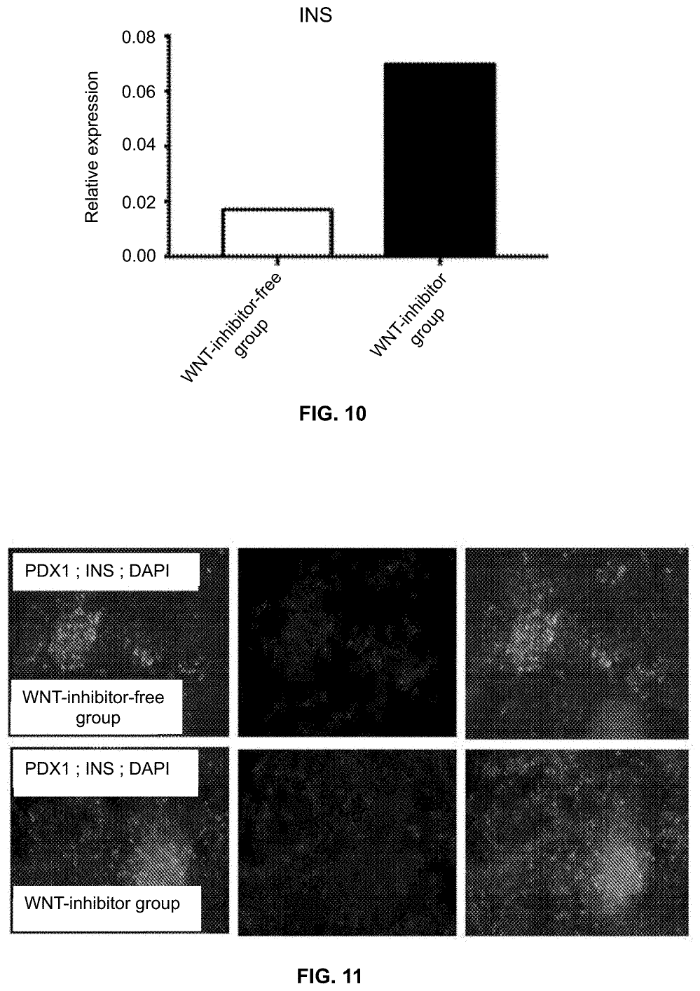

The relative expression of mRNAs of PDX1 and NKX6-1 in the pancreatic progenitor cell stage was detected by means of real-time fluorescent quantitative PCR, and the results were as shown in FIG. 8, wherein in the stage of differentiation from definitive endoderm cells to pancreatic progenitor cells, the mRNAs of the marker PDX1 and its downstream gene NKX6-1 were significantly increased in the pancreatic progenitor cells after the adding of the WNT signaling pathway inhibitor IWR-1; and the relative expression of mRNA of INS in the mature pancreatic islet β cell stage was detected by means of real-time fluorescent quantitative PCR, and the results were as shown in FIG. 10, wherein in the process of further differentiation from pancreatic progenitor cells into mature pancreatic islet β cells, the transcription level of the insulin gene INS in the mature pancreatic islet cell stage was greatly increased.

(4) Immunofluorescence Detection of the Expression of PDX1 Protein in the Pancreatic Progenitor Cell Stage and PDX1/INS Proteins in the Mature Pancreatic Islet β Cell Stage

Cells in a culture plate were washed three times with a phosphate buffered saline (PBS), 3 minutes each time; the cells were fixed with 4% paraformaldehyde at room temperature for 20 minutes, and washed three times with PBS, 3 minutes each time; a blocking solution was prepared in advance (by adding Triton at a final concentration of 0.3% to 9 ml of DPBS, adding donkey serum at a final concentration of 10%, and uniformly mixing); the above-prepared blocking solution was used for blocking and perforation at room temperature for 2 hours; the blocking solution was soaked up, a working concentration of primary antibody prepared with the blocking solution was then added, and the product was left to stand at room temperature for 30 minutes and then placed in a refrigerator at 4° C. overnight; the next day, the product was washed three times with PBS, 3 minutes each time; a fluorescent secondary antibody of donkey serum was added and incubation was carried out at room temperature for 2 hours; in the same way, the product was washed three times with PBS, 3 minutes each time; the product was incubated with a working solution of the nuclear dye DAPI at room temperature for 10 minutes, and washed three times with PBS, 3 minutes each time; then a fluorescence microscope was used for observation and data were collected. The primary antibodies used in this study were as follows: human PDX-1/IPF1 antibody (AF2419, 1:1000, RD); Anti-PDX1 (ab47267, 1:200, Abcam); Anti-PDX1 (ab47383, 1:1000, Abcam); and insulin antibody (SC-29062, 1:100, SantaCruz). The fluorescent secondary antibodies used in this study were donkey anti-goat TRITC fluorescent dye (705-025-147, 1:200, Jackson ImmunoResearch); donkey anti-goat FITC fluorescent dye (705-095-003, 1:200, Jackson ImmunoResearch); donkey anti-goat 488 fluorescent dye (705-545-147, 1:200, Jackson ImmunoResearch); donkey anti-rabbit TRITC fluorescent dye (711-025-152, 1:200, Jackson ImmunoResearch); donkey anti-rabbit 488 fluorescent dye (705-095-152, 1:200, Jackson ImmunoResearch); donkey anti-mouse FITC fluorescent dye (715-095-150, 1:200, Jackson ImmunoResearch); and nuclear dye DAPI (Roche, USA).

The expression of PDX1 protein in the pancreatic progenitor cell stage was detected by means of immunofluorescence, and the results were as shown in FIG. 9, wherein PDX1-positive pancreatic progenitor cells were marked by green fluorescence, and blue DAPI indicated the total cell number of pancreatic progenitor cells. It could be seen that the adding of the WNT signaling pathway inhibitor did not affect the cell growth, and the PDX1-positive pancreatic progenitor cells were significantly increased after the WNT signaling pathway inhibitor IWR-1 was added.

The expression of PDX1/INS proteins in the mature pancreatic islet β cell stage was detected by means of immunofluorescence, and the results were as shown in FIG. 11, wherein PDX1-positive pancreatic islet β cells were marked by green fluorescence, INS-positive pancreatic islet β cells were marked by red fluorescence, and blue DAPI indicated the total cell number of pancreatic islet β cells. It could be seen that after the adding of the WNT signaling pathway inhibitor IWR-1 in the pancreatic progenitor cell stage, the number of insulin INS-positive and PDX1-co-stained pancreatic islet β cells in the mature pancreatic islet β cell stage was significantly increased.

The results showed that the adding of the staged WNT signaling pathway inhibitor provided by the present disclosure could greatly increase the number of mature pancreatic islet β cells.

### Example 3 Differentiation of Human Induced Pluripotent Stem Cells into Pancreatic Progenitor Cells and Mature Pancreatic Islet β Cells by Induction of the WNT Signaling Pathway Inhibitor XAV-939

(1) Cell Differentiation

1) Differentiation of Human Induced Pluripotent Stem Cells into Definitive Endoderm Cells:

a. definitive endoderm stage medium 1 was prepared, and the human induced pluripotent stem cells were cultured in the medium in a carbon dioxide incubator at 37 degrees Celsius for 1 day; and

b. definitive endoderm stage medium 2 was prepared, the definitive endoderm stage medium 1 in above-mentioned step a was replaced with the definitive endoderm stage medium 2, and the cells were cultured in a carbon dioxide incubator at 37 degrees Celsius for 3 days, during which the medium was replaced every day, wherein the composition of the definitive endoderm stage medium 1 was: a basic medium was prepared by mixing an IMDM medium and an F12 medium at a ratio of 1:1, and the following components at the working concentrations were further included: 0.2% of bovine serum albumin (BSA), 1% of penicillin, 1% of streptomycin, 100 ng/ml of recombinant human activin-A (Activin A), and 50 ng/ml of Wnt3a protein, with the concentrations all being final concentrations; and the composition of the definitive endoderm stage medium 2 was: a basic medium was prepared by mixing an IMDM medium and an F12 medium at a ratio of 1:1, and the following components at the working concentrations were further included: 0.2% of bovine serum albumin (BSA), 1% of penicillin, 1% of streptomycin, and 100 ng/ml of recombinant human activin-A (Activin A), with the concentrations all being final concentrations.

2) Induction of the Differentiation of Definitive Endoderm Cells into Pancreatic Progenitor Cells:

a pancreatic progenitor cell medium was prepared, the medium in step 1) was replaced with the pancreatic progenitor cell medium, and the cells were cultured in a carbon dioxide incubator at 37 degrees Celsius for 5 days, during which the medium was replaced every day, wherein the composition of the pancreatic progenitor cell medium was: with an MCDB131 medium as a basic medium, and the following components at the working concentrations were further included: 0.5% of bovine serum albumin (BSA), 1.5 g/L of sodium bicarbonate, 1×glutamine (GlutaMAX), 10 mM glucose, 0.25 M vitamin C, 1×insulin-transferrin-selenium-ethanolamine additive (ITS-X), 50 ng/ml of fibroblast growth factor (KGF), 0.5 μM SANT1 (with smo as an inhibitor target), 100 nM retinoic acid (TTNPB), 500 nM phorbol 12,13-dibutyrate (PDBu), 2 μM ALK inhibitor (K02288), and 2 μM WNT signaling pathway inhibitor XAV-939 (not added in a control group), with the concentrations all being final concentrations.

3) Further Differentiation of Pancreatic Progenitor Cells to Obtain Pancreatic Islet β Cells:

a. pancreatic islet β cell medium 1 was prepared, the medium in step 2) was replaced with the pancreatic islet β cell medium 1, and the cells were cultured in a carbon dioxide incubator at 37 degrees Celsius for 5 days, during which the medium was replaced every day; and

b. pancreatic islet β cell medium 2 was prepared, the pancreatic islet β cell medium 1 in above-mentioned step a was replaced with the pancreatic islet β cell medium 2, and the cells were cultured in a carbon dioxide incubator at 37 degrees Celsius for 5 days, during which the medium was replaced every day, wherein the composition of the pancreatic islet β cell medium 1 was: with an MCDB131 medium as a basic medium, and the following components at the working concentrations were further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 2 μM ALK inhibitor (K02288), 1 μM triiodothyronine (T3), 10 μM YO-01027 (Notch signaling pathway inhibitor), and 10 μM zinc sulfate, with the concentrations all being final concentrations; and the composition of the pancreatic islet β cell medium 2 was: with an MCDB131 medium as a basic medium, and the following components at the working concentrations were further included: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 1 μM triiodothyronine (T3), 10 μM Repsox (ALK5 inhibitor), 10 μM vitamin E, 10 μg/ml of heparin sodium, 2 μM R428 (Axl inhibitor), 10 μM zinc sulfate, and 10 mM N-acetyl-L-cysteine (N-cys), with the concentrations all being final concentrations.

(2) Detection of the Expression of PDX1 in the Pancreatic Progenitor Cell Stage by Means of Flow Cytometry

Adherent cells cultured on a pancreatic progenitor cell stage culture plate were washed three times with PBS free of calcium and magnesium ions (i.e., DPBS), the remaining medium was removed, the cells were covered with 0.05% of chymotrypsin-trypsin (Trypsin) and placed in an incubator at 37° C. for 10 minutes of digestion, the digestion was then terminated using DPBS containing 2% fetal bovine serum (FBS), the product was carefully pipetted to form a single cell suspension, the single cell suspension was centrifuged at 3000 rpm for 5 minutes, and the supernatant was removed to leave cell pellets; the cell pellets were resuspended with 2% FBS in DPBS, the product was carefully pipetted until uniformly mixed, and centrifuged at 3000 rpm for 5 minutes, the supernatant was removed to leave cell pellets, and the operation was repeated once again. A perforating agent in a cell transcription factor kit was prepared (prepared immediately before use, and attention should be paid to avoid light), 1 ml of the perforating agent was used for gentle pipetting, the cell pellets were resuspended, and the product was placed on ice in the dark for 40 minutes of perforation; the perforation was terminated using 1 ml of a detergent in the cell transcription factor perforation kit which was prepared in advance, then the product was centrifuged at 3000 rpm for 5 minutes, and the supernatant was removed to leave cell pellets; 1 ml of the detergent was added to the pellets to resuspend the cells, the product was centrifuged at 3000 rpm for 5 minutes, the supernatant was removed to leave cell pellets, and the operation was repeated once again. A primary antibody diluted to a working concentration with the detergent was added, and the product was placed on a rotary mixer at 4° C. overnight; the next day, the product was taken out from 4° C. and rewarmed for 30 minutes, the primary antibody was terminated using 1 ml of the detergent, then the product was centrifuged at 3000 rpm for 5 minutes, and the supernatant was removed to leave cell pellets; 1 ml of the detergent was added to the cell pellet, the product was centrifuged at 3000 rpm for 5 minutes, the supernatant was removed to leave cell pellets, and the operation was repeated once again to leave the pellets, wherein close observation was carried out each time the supernatant was aspirated, and the cell pellets should not be aspirated. A fluorescent secondary antibody diluted with the detergent was added, and the product was placed on a rotary mixer at room temperature and incubated for 2 hours; the secondary antibody was terminated using 1 ml of the detergent, then the product was centrifuged at 3000 rpm for 5 minutes, and the supernatant was removed to leave cell pellets; 1 ml of the detergent was added to the cell pellets, the product was centrifuged at 3000 rpm for 5 minutes, the supernatant was removed to leave cell pellets, and the operation was repeated once again to leave the pellets. The cell pellets were gently pipetted using 200 μl of DPBS and transferred to a flow tube; and an FACSCelesta flow cytometric analyzer was used for sample analysis. The primary antibody used in this study was human PDX-1/IPF1 antibody (AF2419, 1:500, RD); and the secondary antibody used in this study was donkey anti-goat PE fluorescent dye (115-035-003, 1:200, Jackson ImmunoResearch).

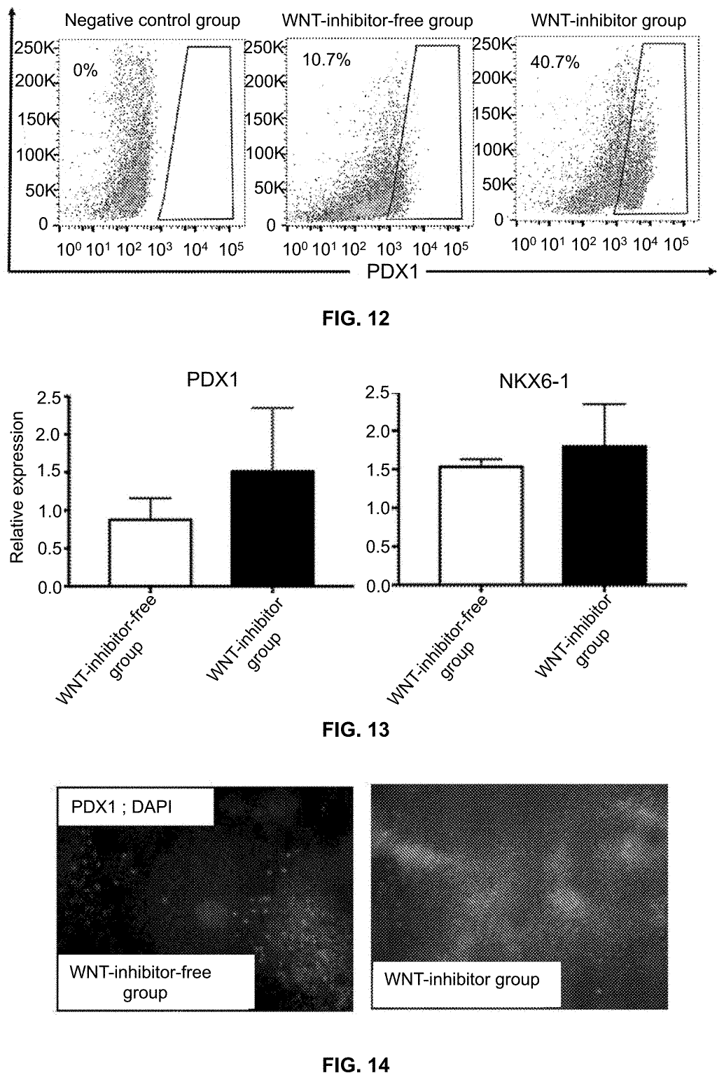

The expression of PDX1 in the pancreatic progenitor cell stage was detected by means of flow cytometry, and the results were as shown in FIG. 12. Compared with a control group in which no WNT signaling pathway inhibitor was added, PDX1-positive pancreatic progenitor cells were significantly increased after the WNT signaling pathway inhibitor XAV-939 was added.

(3) Detection of the Relative Expression of mRNAs of PDX1 and NKX6-1 in the Pancreatic Progenitor Cell Stage and Detection of the Relative Expression of mRNA of INS in the Mature Pancreatic Islet β Cell Stage by Real-Time Fluorescence Quantitative PCR

1) Total RNA Extraction from Cells

Cells cultured in a 24-well plate were collected, and washed twice with 500 μl of DPBS, at first, then covered with 200 μl of chymotrypsin-trypsin (Trypsin), and placed at 37° C. for 5 minutes of digestion. Whether the cells float in pieces was observed, and the digestion was terminated using 500 μl of DMEM/F12 medium, then the product was centrifuged at 1000 rpm for 3 minutes, the supernatant was removed, and cell pellets were collected and added to a new RNase-free centrifuge tube. A lysis solution in a rapid and small-amount extraction kit of total RNA was used, and 350 μl of the lysis solution was added to each tube and then uniformly mixed using a pipette (to avoid bubble generation caused by excessive pipetting), or a vortex mixer was used to break up the cells such that the cells were fully lysed; the product was centrifuged at 14,000×g for 5 minutes at room temperature; after centrifugation, the liquid was taken and added to an equal volume of an RNA binding agent in which anhydrous ethanol was added in advance, then the mixture was added to each centrifuge tube, pipetted five times with a pipette, and the liquid, which had been pipetted until uniformly mixed, was transferred to a high-purification RNA column, which was mounted in a 2 ml collection tube for 1 minute of centrifugation at 12,000×g; the filtrate was discarded, the column was placed back into the collection tube, 500 μl of kit detergent 1 was added to the column, centrifugation was performed at 12,000×g for 1 minute, the filtrate was discarded, the column was placed back into the collection tube, then 500 μl of kit detergent 2 (diluted with ethanol) was added, centrifugation was performed at 12,000×g for 1 minute, and 500 μl of kit detergent 2 was added so as to repeat the operation once again; the filtrate was discarded, and the column was placed back into the collection tube for 2 minutes of centrifugation at 12,000×g; then the column was transferred to a new 1.5 ml centrifuge tube, 50 μl of RNase-free water was added to the center of a column membrane, which was left to stand at room temperature for 2 minutes, followed by 1 minute of centrifugation at 12,000×g, and the above-mentioned operation was repeated once again to improve the RNA elution efficiency. The column was discarded and the RNA was stored in an ultra-low-temperature refrigerator at −80° C.

2) Preparation of cDNA

The concentration and purity of the total RNA extracted above was determined (A260/A280 absorbance ratio >1.8), and a reverse transcription kit from the manufacturer Biotool was used to obtain cDNA, wherein the total RNA was taken and placed on ice, the volume required for the reverse transcription of 1 μg of RNA was calculated according to the concentration, and the corresponding volume of RNA was added; 4 μl of 5×qRT reverse transcription mix was then added, and the rest was made up with RNase-free water to obtain a total volume of 20 μl of a reverse transcription mixture liquid; and the reverse transcription mixture liquid was gently mixed, the uniformly mixed liquid was centrifuged by means of a palm centrifuge to the bottom of a 1.5 ml centrifuge tube, a common Bio-rad PCR instrument was used for reverse transcription under the reaction conditions of 25° C. for 10 min, 42° C. for 30 min and 85° C. for 5 min, and afterwards, the product was stored at −20° C.

3) Detection of the Relative Expression of mRNAs of PDX1, NKX6-1 and INS by SYBR Green Real-Time Fluorescence Quantitative PCR

50 ng of cDNA obtained from the above-mentioned reverse transcription was taken, 5 μl of 2×SYBR Green qPCR mixture, 5 μM of upstream primer and 5 μM of downstream primer were added, and the rest was made up with RNase-free water to a total volume of 10 μl; and CFX384 Bio-rad model quantitative PCR instrument was used for amplification under the reaction conditions of 95° C. for 5 min, 95° C. for 15 s and 60° C. for 30 s, repeated 39 cycles, 95° C. for 15 s, and ending with 65° C. for 15 s. The experimental results were analyzed using a ΔΔCT method with reference to the housekeeping gene GAPDH. Primers used in this study were: GAPDH upstream primer AATGAAGGGGTCATTGATGG, downstream primer AAGGTGAAGGTCGGA GTCAA; PDX1 upstream primer TTAGGATGTGGACGTAATTCCTGTT, downstream primer GGCCACTGT GCTTGTCTTCA; NKX6-1 upstream primer AGACCCACTTTT TCCGGACA, downstream primer CCAACGAATAGGCCAAACGA; INSMLIN upstream primer GCAGCCTTTGTGAACCAACAC, and downstream primer CCCCG CACACTAGGTAGAGA.

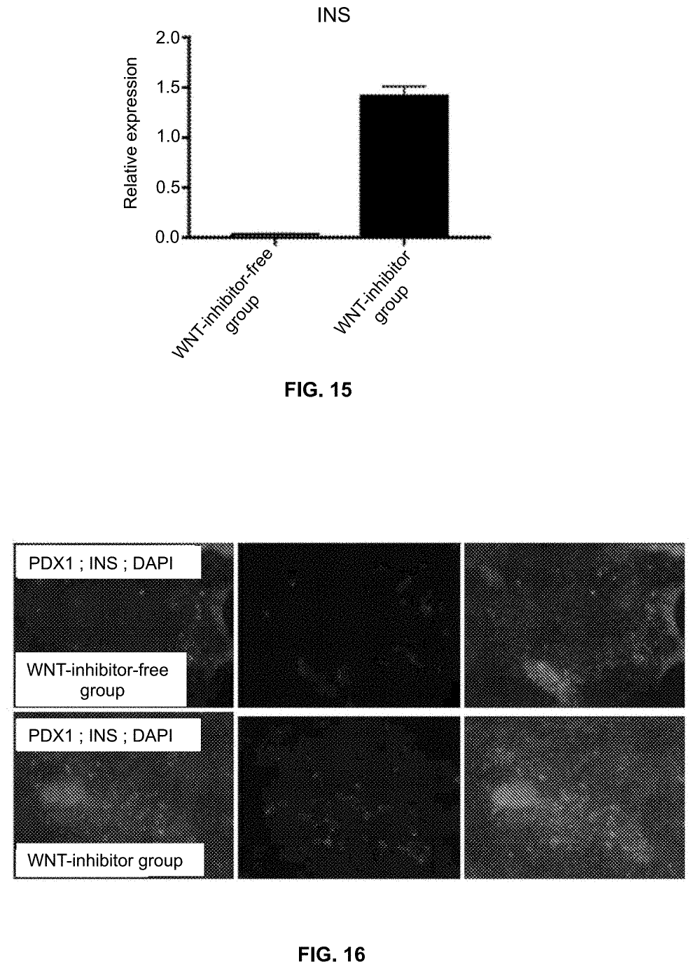

The relative expression of mRNAs of PDX1 and NKX6-1 in the pancreatic progenitor cell stage was detected by means of real-time fluorescent quantitative PCR, and the results were as shown in FIG. 13, wherein in the stage of differentiation from definitive endoderm cells to pancreatic progenitor cells, the mRNAs of the marker PDX1 and its downstream gene NKX6-1 were significantly increased in the pancreatic progenitor cells after the adding of the WNT signaling pathway inhibitor XAV-939; and the relative expression of mRNA of INS in the mature pancreatic islet β cell stage was detected by means of real-time fluorescent quantitative PCR, and the results were as shown in FIG. 15, wherein in the subsequent process of further differentiation from pancreatic progenitor cells into mature pancreatic islet β cells, the transcription level of the insulin gene INS in the mature pancreatic islet β cell stage was greatly increased.

(4) Immunofluorescence detection of the expression of PDX1 protein in the pancreatic progenitor cell stage and PDX1/INS proteins in the mature pancreatic islet β cell stage

Cells in a culture plate were washed three times with a phosphate buffered saline (PBS), 3 minutes each time; the cells were fixed with 4% paraformaldehyde at room temperature for 20 minutes, and washed three times with PBS, 3 minutes each time; a blocking solution was prepared in advance (by adding Triton at a final concentration of 0.3% to 9 ml of DPBS, adding donkey serum at a final concentration of 10%, and uniformly mixing); the above-prepared blocking solution was used for blocking and perforation at room temperature for 2 hours; the blocking solution was soaked up, a working concentration of primary antibody prepared with the blocking solution was then added, and the product was left to stand at room temperature for 30 minutes and then placed in a refrigerator at 4° C. overnight; the next day, the product was washed three times with PBS, 3 minutes each time; a fluorescent secondary antibody of donkey serum was added and incubation was carried out at room temperature for 2 hours; in the same way, the product was washed three times with PBS, 3 minutes each time; the product was incubated with a working solution of the nuclear dye DAPI at room temperature for 10 minutes, and washed three times with PBS, 3 minutes each time; then a fluorescence microscope was used for observation and data were collected. The primary antibodies used in this study were as follows: human PDX-1/IPF1 antibody (AF2419, 1:1000, RD); Anti-PDX1 (ab47267, 1:200, Abcam); Anti-PDX1 (ab47383, 1:1000, Abcam); and insulin antibody (SC-29062, 1:100, SantaCruz). The fluorescent secondary antibodies used in this study were donkey anti-goat TRITC fluorescent dye (705-025-147, 1:200, Jackson ImmunoResearch); donkey anti-goat FITC fluorescent dye (705-095-003, 1:200, Jackson ImmunoResearch); donkey anti-goat 488 fluorescent dye (705-545-147, 1:200, Jackson ImmunoResearch); donkey anti-rabbit TRITC fluorescent dye (711-025-152, 1:200, Jackson ImmunoResearch); donkey anti-rabbit 488 fluorescent dye (705-095-152, 1:200, Jackson ImmunoResearch); donkey anti-mouse FITC fluorescent dye (715-095-150, 1:200, Jackson ImmunoResearch); and nuclear dye DAPI (Roche, USA).

The expression of PDX1 protein in the pancreatic progenitor cell stage was detected by means of immunofluorescence, and the results were as shown in FIG. 14, wherein PDX1-positive pancreatic progenitor cells were marked by green fluorescence, and blue DAPI indicated the total cell number of pancreatic progenitor cells. It could be seen that the adding of the WNT signaling pathway inhibitor did not affect the cell growth, and the PDX1-positive pancreatic progenitor cells were significantly increased after the WNT signaling pathway inhibitor XAV-939 was added.

The expression of PDX1/INS proteins in the mature pancreatic islet β cell stage was detected by means of immunofluorescence, and the results were as shown in FIG. 16, wherein PDX1-positive pancreatic islet β cells were marked by green fluorescence, INS-positive pancreatic islet β cells were marked by red fluorescence, and blue DAPI indicated the total cell number of pancreatic islet β cells. It could be seen that after the adding of the WNT signaling pathway inhibitor XAV-939 in the pancreatic progenitor cell stage, the number of insulin INS-positive and PDX1-co-stained pancreatic islet β cells in the mature pancreatic islet β cell stage was significantly increased.

The results showed that the adding of the staged WNT signaling pathway inhibitor provided by the present disclosure could greatly increase the number of mature pancreatic islet β cells.

1: A method of obtaining pancreatic progenitor cells and pancreatic islet β cells by differentiating human pluripotent stem cells, wherein the method comprises the following steps:
1) differentiating the human pluripotent stem cells into definitive endoderm cells, comprising:
a. preparing definitive endoderm stage medium 1, and culturing the human pluripotent stem cells in the medium in a carbon dioxide incubator at 37 degrees Celsius for 1 day; and
b. preparing definitive endoderm stage medium 2, replacing the definitive endoderm stage medium 1 in mentioned-above step a with the definitive endoderm stage medium 2, and culturing the cells in a carbon dioxide incubator at 37 degrees Celsius for 3 days, during which the medium is replaced every day,
wherein the composition of the definitive endoderm stage medium 1 is: an IMDM medium and an F12 medium mixed at a ratio of 1:1 as a basic medium, and further comprising the following components at working concentrations of: 0.2% of bovine serum albumin (BSA), 1% of penicillin, 1% of streptomycin, 100 ng/ml of recombinant human activin-A (Activin A), and 50 ng/ml of Wnt3a protein, with the working concentrations all being final concentrations; and
wherein the composition of the definitive endoderm stage medium 2 is: the IMDM medium and the F12 medium mixed at a ratio of 1:1, and further comprising the following components at the working concentrations arc of: 0.2% of bovine serum albumin (BSA), 1% of penicillin, 1% of streptomycin, and 100 ng/ml of recombinant human activin-A (Activin A), with the working concentrations all being final concentrations;
2) inducing differentiation of definitive endoderm cells into pancreatic progenitor cells, comprising:
preparing a pancreatic progenitor cell medium, replacing the medium in step 1) with the pancreatic progenitor cell medium, and culturing the cells in a carbon dioxide incubator at 37 degrees Celsius for 5 days, during which the medium is replaced every day,
wherein the composition of the pancreatic progenitor cell medium is: an MCDB131 medium as a basic medium, and further comprising the following components at the working concentrations of: 0.5% of bovine serum albumin (BSA), 1.5 g/L of sodium bicarbonate, 1×glutamine (GlutaMAX), 10 mM glucose, 0.25 M vitamin C, 1×insulin-transferrin-selenium-ethanolamine additive (ITS-X), 50 ng/ml of fibroblast growth factor 7 (KGF), 0.5 μM SANT1 (with smo as an inhibitor target), 100 nM retinoic acid (TTNPB), 500 nM phorbol 12,13-dibutyrate (PDBu), 2 μM ALK inhibitor (K02288), and a WNT signaling pathway inhibitor, with the working concentrations all being final concentrations; and
3) differentiating further the pancreatic progenitor cells to obtain pancreatic islet β cells, comprising:
a. preparing pancreatic islet β cell medium 1, replacing the medium in step 2) with the pancreatic islet β cell medium 1, and culturing the cells in a carbon dioxide incubator at 37 degrees Celsius for 5 days, during which the medium is replaced every day; and
b. preparing pancreatic islet β cell medium 2, replacing the pancreatic islet β cell medium 1 in the above-mentioned step a with the pancreatic islet β cell medium 2, and culturing the cells in a carbon dioxide incubator at 37 degrees Celsius for 5 days, during which the medium is replaced every day,
wherein the composition of the pancreatic islet β cell medium 1 is: the MCDB131 medium as the basic medium, and further comprising the following components at the working concentrations of: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 2 μM ALK inhibitor (K02288), 1 μM triiodothyronine (T3), 10 μM YO-01027 (Notch signaling pathway inhibitor), and 10 μM zinc sulfate, with the working concentrations all being final concentrations; and
wherein the composition of the pancreatic islet β cell medium 2 is: the MCDB131 medium as the basic medium, and further comprising the following components at the working concentrations of: 20 mM glucose, 2% of bovine serum albumin (BSA), 1.5 g/L of sodium bicarbonate, 1×glutamine (GlutaMAX), 0.05 mM vitamin C, 1×insulin-transferrin-selenium-ethanolamine additive (ITS-X), 1 μM triiodothyronine (T3), 10 μM Repsox (ALK5 inhibitor), 10 μM vitamin E, 10 μg/ml of heparin sodium, 2 μM R428 (Axl inhibitor), 10 μM zinc sulfate, and 10 mM N-acetyl-L-cysteine (N-cys), with the working concentrations all being final concentrations.

2: The method according to claim 1, wherein the human pluripotent stem cells are human embryonic stem cells.

3: The method according to claim 1, wherein the human pluripotent stem cells are human induced pluripotent stem cells.

4: The method according to claim 2, wherein the WNT signaling pathway inhibitor in step 2) is XAV-939 or IWR-1.

5: The method according to claim 3, wherein the WNT signaling pathway inhibitor in step 2) is XAV-939.

6: The method according to claim 1, wherein the concentration of the WNT signaling pathway inhibitor in step 2) is 2 μM.


SEQUENCE LISTING (8 sequences)

SEQ ID NO: 1 - DNA, 20 bp, Artificial Sequence
  Upstream primer for GAPDH housekeeping gene
aatgaaggggtcattgatgg

SEQ ID NO: 2 - DNA, 20 bp, Artificial Sequence
  Downstream primer for GAPDH housekeeping gene
aaggtgaaggtcggagtcaa

SEQ ID NO: 3 - DNA, 25 bp, Artificial Sequence
  Upstream primer for pancreatic duodenal homeobox 1 (PDX1) gene
ttaggatgtggacgtaattcctgtt

SEQ ID NO: 4 - DNA, 20 bp, Artificial Sequence
  Downstream primer for pancreatic duodenal homeobox 1 (PDX1) gene
ggccactgtgcttgtcttca

SEQ ID NO: 5 - DNA, 20 bp, Artificial Sequence
  Upstream primer for NK6 homeobox protein 1 (NKX6-1) gene
agacccactttttccggaca

SEQ ID NO: 6 - DNA, 20 bp, Artificial Sequence
  Downstream primer for NK6 homeobox protein 1 (NKX6-1) gene
ccaacgaataggccaaacga

SEQ ID NO: 7 - DNA, 21 bp, Artificial Sequence
  Upstream primer for insulin (INS) gene
gcagcctttgtgaaccaacac

SEQ ID NO: 8 - DNA, 20 bp, Artificial Sequence
  Downstream primer for insulin (INS) gene
ccccgcacactaggtagaga